(19) United States
(12) Patent Application Publication
(10) Pub. No.: US 2021/0072127 A1
(43) Pub. Date: Mar. 11, 2021

(54) NON-DESTRUCTIVE SAMPLING DEVICE FOR EXTRACTION OF A MARKER

(71) Applicant: UNIVERSITE DE LIEGE, ANGLEUR (BE)

(72) Inventors: Vincent CASTRONOVO, LIEGE (BE); Carl EMMERECHTS, HUY (BE)

(21) Appl. No.: 16/644,811

(22) PCT Filed: May 19, 2018
(86) PCT No.: PCT/EP2018/063244

§ 371 Date: Mar. 5, 2020

(30) Foreign Application Priority Data
Sep. 6, 2017 (BE) BE 201705633

(51) Int. Cl. G01N 1/40; G01N 33/574

(52) U.S. Cl. CPC G01N 1/4005; G01N 33/57488

(57) ABSTRACT

Method and device for extraction of markers, including: introducing a sample into a removable support basket placed in the housing of a cell of an extraction device provided with a flexible membrane and immersing the tissue sample in a liquid previously introduced into the housing, followed by hermetic closing of the extraction cell and application of a pressure cycle on the flexible membrane for a period of 1 to 5 minutes at a frequency between 1 and 2 Hz. Then, total or partial recovery of the liquid immersing the sample after the pressure cycle and detection of the markers present in the liquid.

### FIELD

The present invention relates to a non-destructive sampling method, device and apparatus, for the extraction of markers from a sample.

More particularly, the invention relates to a method, a device and an apparatus for the extraction of biomarkers from biological samples and to the use thereof for diagnosis in the early stage of a disease.

### BACKGROUND

The extraction of markers at low concentration in a sample, regardless of the origin thereof, proves to be problematic when the integrity of the sample must be maintained.

In particular, the search for biomarkers or biological markers capable of detecting and evaluating the early stage of a disease such as cancer, or predicting the resistance and the response to treatment, remains a challenge.

Apparently, the non-invasive search for biomarkers is carried out essentially starting from blood, serum, urine or saliva samples. In these liquids, the biomarkers are diluted up to a one billion times compared with the concentration in the disease tissue, thereby making it virtually impossible to detect them starting from these media.

The second limitation is the inaccessibility of a sample of tissues such as solid biopsies, which are primarily intended in their entirety for analysis for diagnosis. Thus, for obvious ethical reasons, this precious human material is not accessible for the search for new biomarkers that are useful and relevant. Said biomarkers are intended in their entirety for complete histological evaluation in clinical testing. These samples are, moreover, taken in very small amounts, about 1 to 125 mm³.

The detection of biomarkers is nevertheless essential for being able to provide an individualized diagnosis and enabling the best therapy possible to be selected.

### SUMMARY

The objective of the present invention is to overcome the problem of low availability of markers when they are extracted from samples of an origin whatsoever, when the integrity of the sample must be respected.

More particularly, the objective of the present invention is to overcome the problem encountered during the use of a liquid biopsy such as blood, urine or saliva with too low a concentration of biomarkers, but also to overcome a lack of availability of solid biopsy.

In a first aspect, the invention relates to an easy and rapid method for extracting a marker from a sample, comprising the following steps:
introducing a sample into a disposable support basket placed in the housing of a cell of an extraction device provided with a flexible membrane and
immersing the sample in a liquid previously introduced into the housing of the cell;
hermetically enclosing the extraction cell after immersion of the sample in the liquid, and applying a pressure cycle on the flexible membrane for a period of 1 to 5 minutes, preferably 3 minutes, at a frequency of between 1 and 2 Hz , preferably 1.5 Hz; then
totally or partially recovering the liquid immersing the sample after application of the pressure cycle on the membrane, and detecting the markers present in the liquid.

The hydrostatic pressure applied on the membrane of the device is between 2 and 5 bar, and is preferably 2 bar.

A pressure cycle means that a pressure alternating between maximum and minimum pressure is applied for a given period of time at a given frequency.

The term “sample” is intended to mean any type of sample in the solid state, derived from foods, from plant tissue, from biological tissues, from a mineral composition or the like, capable of comprising one or more markers at low concentration in the sample. More particularly, the biological tissue sample or biological sample is generally derived from a surgical specimen, also called biopsy, taken for example by means of a needle or of a scalpel. The tissue sampling may take place in any organ or element of the human or animal body, such as for example the lung, breast, liver, kidney, uterus, prostrate, bone marrow, muscle, colon, bronchus and the like. The tissue sample may be derived from a cancerous tumor.

The term “liquid” is intended to mean any medium of low viscosity or of viscosity close to water, capable of solubilizing markers present in a solid sample immersed in the medium. The term “liquid” is intended to mean for example a medium based on water, on alcohol, on oil and the like. An example of an aqueous medium used for a biological sample is a physiological liquid.

The term “physiological liquid” is intended to mean a liquid that is isotonic with blood, or that has the same osmolarity as the main body fluids such as blood, urine or saliva. The physiological liquid is for example composed of distilled water and of sodium chloride diluted to 0.9% of weight/volume of NaCl, i.e. 9 g/l. The physiological liquid may be more complex and may comprise for example potassium chloride (KCl), calcium chloride (CaCl₂), magnesium sulfate (MgSO₄) depending on the tissue under consideration.

Finally, phosphate buffered saline (PBS) is also used with 9 g/l NaCl or other salts such as KCl or CaCl₂ as physiological liquid particularly with samples derived from cancerous tumors.

The pressure cycle on the membrane of the extraction device must make it possible to obtain, from the liquid, in particular from the physiological liquid, a sufficient amount of markers, in particular of biomarkers, extracted from the sample while at the same time avoiding degradation of the sample, in particular the tissue or biopsy sample. The application of a low pressure of between 2 and 5 bar, on the flexible membrane separating the liquid, allows an extrusion, from the sample, of markers present in the sample. In the case of a biological sample, the application of a low pressure between 2 and 5 bar, on the flexible membrane separating the liquid, allows an extrusion, from the tissue sample, of a fluid interstitial to the cells of the tissue which contains the biomarkers of interest. The pressure cycle must nevertheless make it possible to maintain the morphology and the antigenicity of the tissue sample for the purposes of subsequent analyses.

After having been subjected to a cyclical pressure for a short period of 1 to 5 minutes, the pressure is brought back to atmospheric pressure and the cell is disconnected from the piston cylinder. A fraction or all of the liquid, in particular the physiological liquid, is extracted from the cell for detection of markers or biomarkers, while the basket which has the sample is removed from the cell so as to allow other subsequent analyses on the sample having kept its initial integrity intact. In the case of a sample of biological tissues, the latter will be subjected to subsequent clinical analyses.

The term “marker” is intended to mean any substance present at low concentration in the solid sample and capable of being solubilized in the sample immersion liquid. A marker may for example be a biological compound, which may or may not be toxic, present in a sample of animal or plant origin or a solid food. A marker may also be a biomarker.

The term “biomarker” is intended to mean not only proteins, but also circulating DNA, exosomes, miRNA, metabolites and equivalent molecules which are in the liquid interstitial to the cells of the tissue under consideration.

The method according to the invention has the advantage that the sample is recovered with its morphological and chemical or biochemical integrity after extraction and detection of the markers or biomarkers.

The method for extracting markers or biomarkers has the advantage of being easy and rapid. It only takes a few minutes and does not cause any delay in the subsequent tests on the sample, such as clinical tests in the case of samples of biological tissues. If a portion of the physiological liquid is kept in the extraction cell, it can be stored at low temperature in a cryopreservation liquid such as liquid nitrogen.

In a second aspect, the invention relates to a device for extracting a marker, which, for the understanding of the invention, is illustrated in FIGS. 1 to 7.

### BRIEF DESCRIPTION OF THE DRAWINGS

FIG. 1: diagram of the extraction cell according to the invention.

FIG. 2: step of introducing the sample into the extraction cell.

FIG. 3: step of hermetically closing the extraction cell by means of a cap consisting of two parts.

FIG. 4: illustration of the system of pressure on the liquid in which the sample is immersed.

FIG. 5: step of removing, after the pressure cycle, the support basket comprising the sample.

FIG. 6: diagram of the apparatus comprising the extraction device according to the invention.

FIG. 7: example of an extraction cell and of an apparatus according to the invention.

FIGS. 8: comparison of immunohistochemical (IHC) analysis on samples of biological tissues having undergone the extraction of biomarkers according to the invention and reference samples not subjected to the extraction.

FIG. 9A: Colorectal cancer-comparison of immunohistochemical (IHC) analysis on samples of biological tissues having undergone the extraction of biomarkers according to the invention and reference samples not subjected to the extraction.

FIG. 9B: Liver metastases- comparison of immunohistochemical (IHC) analysis on samples of biological tissues having undergone the extraction of biomarkers according to the invention and reference samples not subjected to the extraction.

FIG. 10: example of use of the device according to the invention for detecting protein markers.

FIG. 11: example of use of the device according to the invention for detecting metabolic markers.

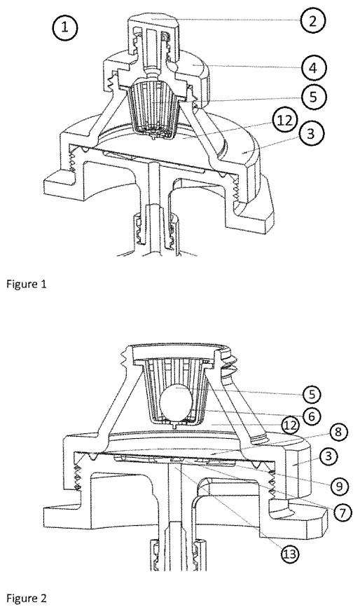

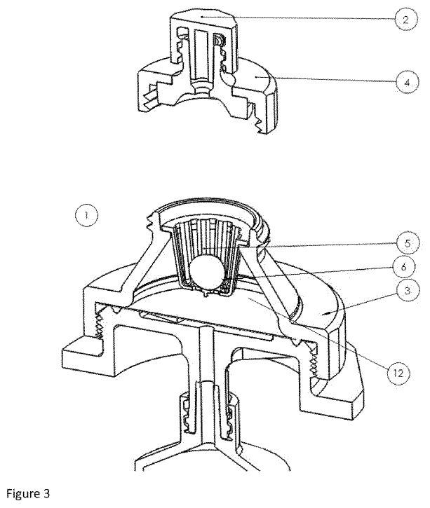

The figures serve to illustrate the embodiments of the invention but do not limit the invention.

### DETAILED DESCRIPTION

The extraction device (1) comprises an extraction cell (3) provided with a housing (12) for receiving a removable basket (5), said basket (5) being configured to immerse a sample (6) in a liquid previously introduced into the housing (12).

Each element belonging to the extraction device is manufactured by injection molding.

In the case of a device for biological use, the manufacturing is carried out in a cleanroom in order to meet the requirements for manufacturing a medical device. Once manufactured, the elements of the extraction device are washed by means of an organic solvent such as isopropanol in an ultrasonic bath. The level of sterilization of the elements thus manufactured must comply with the DIN EN ISO 17665 standard for medical devices and must be such that no trace of DNA is identifiable.

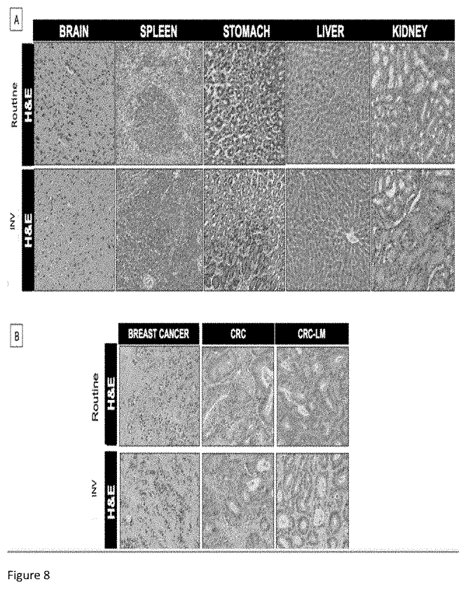

The extraction cell is preferably made of a transparent polymeric material such as polymethacrylate as illustrated in FIG. 7.

The extraction device (1) also comprises a cavity (7) separated from the housing (12) of the extraction cell by means of a flexible membrane (8).

The assembly consisting of the cavity and the flexible membrane allows a compression or a dilation of the liquid and of the tissue sample present in the hermetically closed extraction cell.

The volume of the cavity is between 3 and 10 ml, preferably 4.5 ml.

The membrane (8) is made of flexible polymeric material and withstands a deformation of 50% to 200% of its surface area. The membrane must withstand the deformation during the compression of the liquid or the dilation of the liquid during storage of the extraction cell (3) in liquid nitrogen for example. Preferably, the flexibility of the membrane allows a degree of deformation of 1 to 2 times its surface area.

The membrane is preferably made of silicone with a thickness which can range from 50 to 1000 microns, with a preference of 100 microns.

The flexible membrane, preferably made of silicone, is attached between two assembly parts and is placed for example at the base of the extraction cell (3), by means of a V-shaped groove. The two assembly parts may optionally be optionally separated from one another.

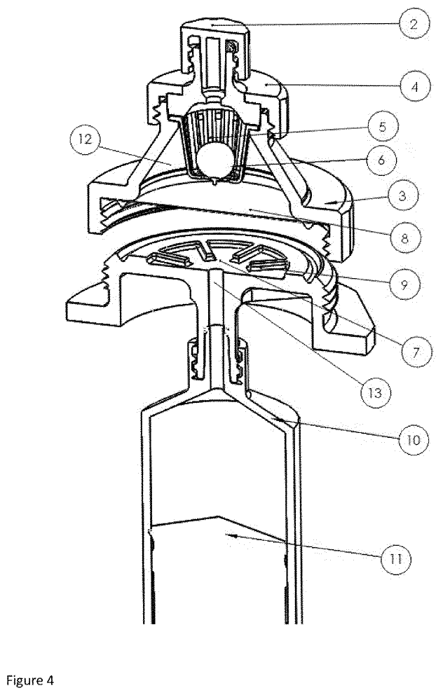

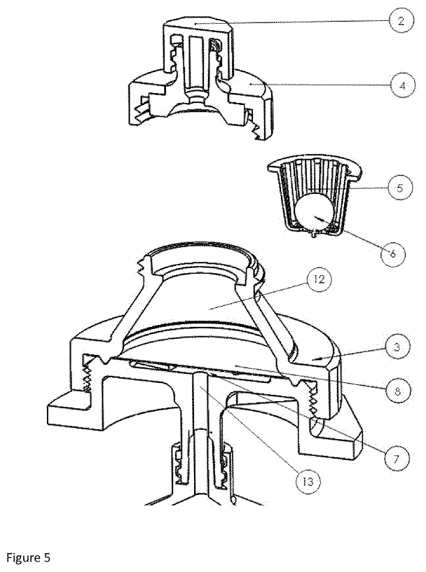

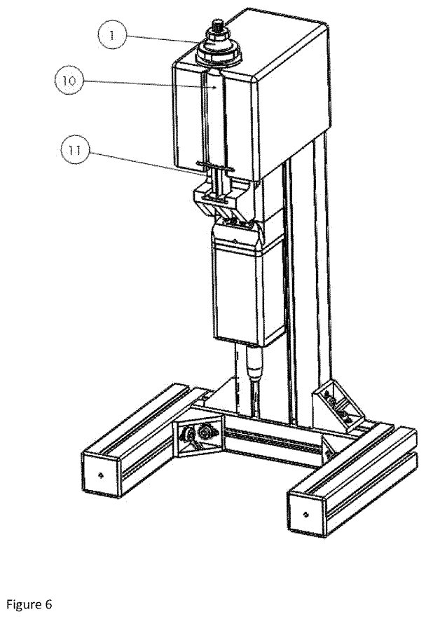

The extraction device (1), as illustrated in FIG. 3, also comprises a leaktight cap, placed on the extraction cell (3) so as to attach the removable support basket (5) in the housing (12) and to keep the liquid inside the housing (12) during the pressure cycle.

According to one preferred embodiment, the leaktight cap comprises two portions, an upper portion (2) or Luer Lock cap placed on a lower portion (4) configured to allow the extraction of the liquid from the cell and to maintain the leaktightness of the extraction cell during the pressure cycle.

The lower portion of the cap is preferably screwed onto the extraction cell by means of a cone-to-cone system thus allowing the extraction cell to be leaktight.

The support basket (5) is manufactured by injection molding, also made of plastic, preferably polypropylene. In one preferred embodiment, the support basket is disposable.

Preferably, the basket is provided with vertical ribs allowing the immersion of the tissue sample in the physiological liquid. The ribs of the container must have a width suitable for the size of the sample so as to allow the sample to remain in the basket while at the same time being immersed in the physiological liquid.

The volume of the support basket (5) in the housing (12) of the extraction cell (3) represents between 10% and 50%, preferably 20%, of the volume of the housing inside the extraction cell. The volume of the basket is also linked to the size of the sample. According to another preferred embodiment, the extraction device comprises a cylinder (10) provided with a piston (11), said cylinder being connected to the cavity (9) at the orifice (13). The cavity-membrane assembly makes it possible to obtain a uniform distribution of the pressure on the liquid and a better marker extraction performance.

Preferably, the volume of the cavity and of the cylinder above the piston is filled with air, but any other inert gas may also be used, such as nitrogen, argon or equivalent. The total volume of the cavity and the cylinder filled with air can range from 20 to 100 ml, but is preferably about 30 ml.

In an even more preferred embodiment of the device according to the invention, the cylinder provided with the piston is a disposable medical syringe.

The cylinder is generally vertically connected to the extrusion cell, but any other configuration, such as for example a horizontal configuration, is also possible.

According to another preferred embodiment, the cavity comprises one or more stiffeners (9). When the stiffeners are multiple of 2, they are preferably arranged in a star pattern around the cylinder (10) connection orifice (13). The use of a stiffener makes it possible to advantageously avoid the membrane sticking to the wall of the cavity.

In the case of a device for biological use, the liquid will preferably be a physiological liquid for the extraction of biomarkers from a biological tissue sample immersed in this physiological liquid.

In a 3rd aspect, the present invention also relates to a device for extracting biomarkers for early diagnosis of diseases at a primary stage or for treating a disease, more particularly treating cancer.

In a 4th aspect, the present invention also relates to a use of the device for detecting molecular metabolic and protein biomarkers from a sample of tissues or solid biopsy.

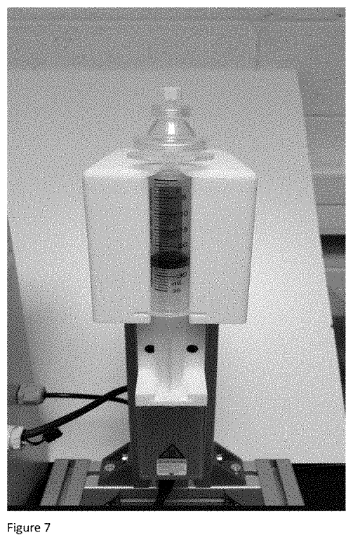

Finally, in a 5th aspect, the present invention relates to an apparatus as illustrated in FIG. 6 integrating the extraction device and also means for actuating the pressure piston, such as for example a jack. The apparatus may also comprise an automatic parameter control module.

FIG. 7 represents an example of an apparatus comprising the extraction cell and also a 30 ml surgical syringe as piston cylinder.

### EXAMPLES

The method, the device and the apparatus were tested on a large series of biological tissue samples obtained from primary colorectal (CRC) tumors and from liver metastasis (CRC-LM). The liquid extruded from the samples is a unique material for detecting biomarkers and does not in any way interfere in the morphology of the tissue for the subsequent clinical tests, as illustrated below.

### 1. Application of the Extraction Method According to the Invention

Freshly surgically removed biopsies were collected immediately after operation, cut up into samples of 3 mm³ and placed in a 4.5 ml cell. Four ml of hypertonic PBS buffer with NaCl, 4.5 g in 500 ml (Weestburg), were added to the cell at the same time as the sample in the basket. Independently, the piston of the syringe is then moved as far as the 15 ml marking. The syringe is then connected to the extraction cell. The sample is subsequently subjected to an alternating pressure at a frequency of 1 Hz for one minute by movement of the piston in the syringe between the 15 ml marking and the 7.5 ml marking. During measurements carried out separately with a connected manometer, it appears that this change in volume corresponds to an effective pressure variation between 2 bar (compression state) and 1 bar (relaxed state) and 1 bar at the initial point when the piston is at the 15 ml marking. The procedure is repeated 3 times for each example. The extraction fluids were then stored at −20° C. The entire procedure lasted 3 minutes and the sample is restored for clinical evaluation. A similar protocol was applied to samples from human breast cancer tumors, primary colorectal cancer and liver metastasis tumors and colon tumors, and also on their adjacent normal counterparts.

### 2. Comparison of the Immunohistochemistry (IHC) Results on Samples Having/Not Having Been Subjected to the Extraction Method According to the Invention

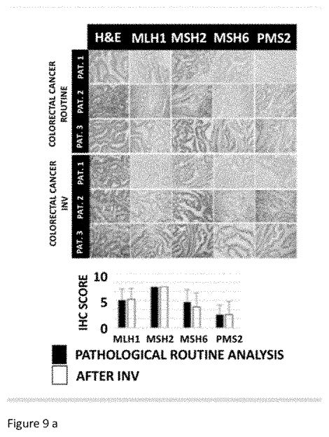

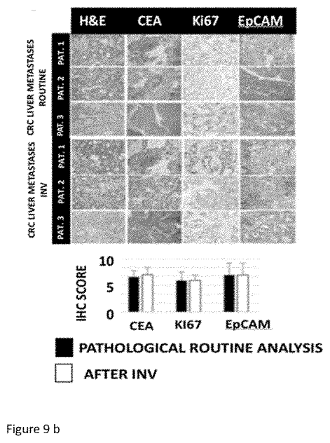

In order to verify that the method according to the invention does not change the morphology and antigenicity of the tissue samples, a comparative analysis of various human and mouse tissues was subjected to the method. Each tissue sample was divided into two; on the one hand, for routine pathological analysis and, on the other hand, for the extraction method according the invention, also referred to as INV in FIGS. 8 and 9. The samples for the routine analysis and/or the extraction method according to the invention are subjected to the same immunohistochemistry (IHC) according to the protocol known to those skilled in the art and described by A. Bellahcene and V. Castronovo in Am. J Pathol 1955 January; 146 (1) : 95-100.

In FIG. 8, primary colorectal tumor samples (n=10, left-hand panel) and metastasized samples liver (n=10, right-hand panel) were subjected to the routine clinical analysis and to the extraction method according to the invention, followed by hematoxylin/eosin (H&E) analysis and immunolabeling for the markers identified (MLH1, MSH2, MSH6, PMS2). The quantitative evaluation for each marker was carried out according to the methods known to those skilled in the art and described by D. Waltregny et al., in J Natl Cancer Inst. 1998 July 1; 90 (13):1000-8.

Samples from 3 patients were collected in each case. Each IHC image was evaluated for its intensity using the following scale (0 for no trace, 1 for weak, 2 for moderate and 3 for strong). The tissues were also evaluated for the extent of their positivity using the scale 1=0-33%, 2=33-66%, 3=66-100%. The values obtained by each of the two evaluations are multiplied to give the HIC score reproduced in FIG. 8.

In FIG. 9A, samples of mouse tissues exhibiting a breast cancer (n=3), a colorectal cancer (CRC; n=3) or in FIG. 9B, a metastatic liver cancer (CRC-LM; n=3) were subjected either to the routine test or to the extraction method followed by the hematoxylin/eosin (H&E) analysis.

As illustrated in FIGS. 8, 9A and 9B, no significant difference was demonstrated regarding the structure of the tissue (H&E) and the intensity of the markers chosen.

### 3. Use of the Device for Identifying Protein and Metabolic Markers in Human Breast Cancer

Ten specimens of breast cancer and their neighboring non-tumor tissues were treated according to the protocol described in example 1, then subjected to the device and method according to the invention. The liquid obtained after application of the extraction device was then analyzed by mass spectrometry for the detection of soluble proteins and by nuclear magnetic resonance for the detection of metabolites. The markers identified from the liquid obtained from the cancerous tissues were then compared to those of the normal tissues.

### 3.1 Protein Marker Detection

Among the proteins detected in the cancerous tissues, Periostin, comp, Ezrin, Raxidin, Versican and Tenascin are for example identified. These proteins are generally present in the extracellular space of the tissues, as illustrated in table 1 below reproducing Pubmed extracts:

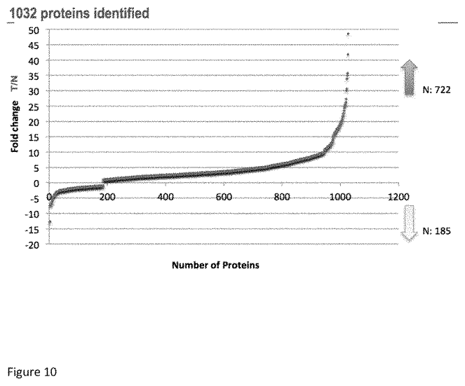

FIG. 10 illustrates the number of protein markers identified in the liquid obtained subsequent to the extraction device and method according to the invention. The markers identified from the liquid in which a cancerous tissue was immersed are compared to those extracted from the normal tissues.

1032 proteins were thus identified from the cancerous tissues and are expressed differentially (722 increased and 185 decreased) in FIG. 10.

### 3.2 Metabolic Marker Detection

The Ingenuity Pathway Analysis was used for the interpretation of the harvested data and their analysis by means of the IPA Core Analysis software.

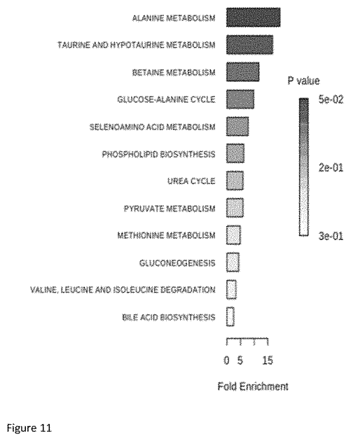

Illustrated in FIG. 11 are the metabolic markers identified according to the extraction method and device according to the invention. An alteration and an increase in the metabolites are observed in the case of the breast cancer. Alanine, taurine and hypotaurine, betaine and glucose alanine are the markers having undergone the greatest up-regulation, as reflected by the P value.

The P value indicates which molecule is significantly associated with reference molecules introduced, this being relative to all the functional molecules characterized. The folding enrichment indicates the statistical significance of the presence of the metabolite in the sample.

1. A method for extracting a marker, comprising:
introducing a sample into a removable support basket placed in the housing of a cell of an extraction device provided with a flexible membrane and
immersing the sample in a liquid previously introduced into the housing;
hermetically closing the extraction cell after immersion of the sample in the liquid and
applying a pressure cycle on the flexible membrane for a period of 1 to 5 minutes, at a frequency of between 1 and 2 Hz;
totally or partially recovering the liquid immersing the sample after the pressure cycle, and detecting the markers present in the physiological liquid;
wherein the pressure applied on the membrane is between 2 and 5 bar.

2. The method as claimed in claim 1, wherein the duration of the pressure cycle is 3 minutes.

3. The method as claimed in claim 1, wherein the sample is a biological tissue sample and the liquid is a physiological liquid for the extraction of biomarkers.

4. The method as claimed in claim 3, wherein the biological tissue sample is extracted from a cancerous tumor.

5. The method as claimed in claim 3, wherein the physiological liquid is a PBS buffer with 9 g/l NaCl.

6. A device for extracting a marker, comprising:
an extraction cell provided with a housing configured to receive a removable support basket, said basket being configured to immerse a sample in a liquid previously introduced into the housing;
a cavity separated from the housing of the extraction cell by a flexible membrane, allowing a compression or a dilation of the liquid; and
a leaktight cap placed on the extraction cell configured to attach the removable support basket in the housing.

7. The device as claimed in claim 6, wherein the leaktight cap comprises two portions, an upper portion or Luer Lock cap placed on a lower part configured to maintain the leaktightness of the extraction cell during the pressure cycle and to allow the extraction of the liquid at the end of the cycle.

8. The device as claimed in claim 6, wherein the device also comprises a piston cylinder connected to the cavity at the orifice.

9. The device as claimed in claim 8, wherein the cavity and the volume of the cylinder above the piston are filled with air.

10. The device as claimed in claim 6, wherein the cavity also comprises at least one stiffener.

11. The device as claimed in claim 10, wherein at least two stiffeners are placed in a star pattern around the cylinder connection orifice.

12. The device as claimed in claim 6, wherein the support basket is provided with vertical ribs allowing the immersion of the sample in the liquid.

13. The device as claimed in claim 12, wherein the ribs of the support basket have a width suitable for the size of the sample.

14. The device as claimed in claim 6, wherein the support basket is made of plastic.

15. The device as claimed in claim 6, wherein the flexibility of the membrane allows a degree of deformation of 1 to 2 times its surface area.

16. The device as claimed in claim 8, wherein the cylinder is vertically connected to the extrusion cell.

17. The device as claimed in claim 8, wherein the cylinder provided with the piston is a disposal syringe.

18. The extraction device as claimed in claim 6, wherein the sample is a biological tissue sample and the liquid is a physiological liquid for the extraction of biomarkers.

19. A method of early diagnosing cancer at the primary stage comprising extracting a biomarker using the device as claimed in claim 18.

20. A method of treating cancer comprising extracting a biomarker using the device as claimed in claim 18.

21. A method of detecting molecular metabolic and protein biomarkers from a solid biopsy comprising using the the device as claimed in claim 18.

22. An apparatus comprising the device for extracting markers as claimed in claim 8, and an actuator configured to move the piston.

23. The apparatus as claimed in claim 22, wherein the actuator is a jack.
